(19) United States
(12) Patent Application Publication
(10) Pub. No.: US 2017/0013898 A1
(43) Pub. Date: Jan. 19, 2017

(54) Restraint Mitten

(71) Applicant: Louise Lewis, RIVIERA BEACH, FL (US)

(72) Inventor: Louise Lewis, RIVIERA BEACH, FL (US)

(21) Appl. No.: 14/799,752

(22) Filed: Jul. 15, 2015

(51) Int. Cl. A41D 19/015; A61F 5/37; A41D 19/01

(52) U.S. Cl. CPC A41D 19/01582; A41D 19/01; A61F 5/37

(57) ABSTRACT

A restraint mitten for hospital and other patients that restricts the movement of his or her fingers. The restraint mitten includes a hand covering having an opening thereon for insertion of the user's hand. The lower portion of the hand covering is padded and the upper portion of the hand covering includes a mesh-like material that allows the user's hand to be visible when positioned inside the mitten. The opening of the hand covering includes an adjustable strap therearound for tightening the mitten onto the user's wrist. The tip of the hand covering includes a movable flap thereon for removably covering the user's fingers so as to allow access to the user's fingers if so desired. The restraint mitten limits the dexterity of the patient's hand, preventing him or her from removing tubes or lines that may be detrimental to the patient's recovery.

### BACKGROUND OF THE INVENTION

### Field of the Invention

The present invention relates to a restraint mitten. More specifically, the present invention provides a restraint mitten for restricting the movement of a patient's fingers to prevent the removal of tubes or lines that could delay recovery and be detrimental to his or her health. The restraint mitten includes a hand covering having an opening providing access to the interior volume thereof, wherein the lower portion of the hand covering comprises a padded material and the upper portion comprises a mesh-like material. An adjustable strap is positioned around the opening of the hand covering, and a flap is movably positioned over the fingertips of the user to provide access thereto.

Hospital patients and others receiving medical care often require tubes and lines to be placed on the user's body. However, patients may accidentally or unintentionally remove intravenous lines which can negatively affect their health and treatment. For example, patients may accidentally remove intravenous lines if they move around in bed, such as while asleep or sedated. Healthcare professionals may not immediately notice that the patient has removed a tube or line, causing the patient to fail to receive the necessary treatments. This can negatively affect the health and well-being of the patient who is in need of the treatments. Thus, a device for inhibiting a patient from removing tubes and lines from his or her body is desired.

Devices in the prior art are known that relate to restraint mitts. Some such devices comprise a mitt having an interior volume in which all of a user's fingers can be positioned, and that include a wrist strap for securing the mitt in position, such as U.S. Patent Publication Number 2012/0192877, U.S. Patent Publication Number 2009/0260638, and U.S. Pat. No. 3,741,207. Other patents provide hand and wrist restraints and cushioned coverings, such as U.S. Pat. No. 5,604,933 and U.S. Pat. No. 5,257,418. Such devices fail to provide a restraint mitten having a padded lower portion and a mesh upper portion, wherein the upper portion includes a movable flap for removably covering the user's fingers disposed inside of the restraint mitten.

In light of the devices disclosed in the prior art, it is submitted that the present invention substantially diverges in design elements from the prior art and consequently it is clear that there is a need in the art for an improvement to existing mitten restraint devices. In this regard, the instant invention substantially fulfills these needs.

### SUMMARY OF THE INVENTION

In view of the foregoing disadvantages inherent in the known types of mitten restraint devices now present in the prior art, the present invention provides a new mitten restraint device wherein the same can be utilized for providing convenience for the user when restraining a hand of a user to prevent the user from removing intravenous lines or for limiting the movement of the user's hand or fingers.

It is therefore an object of the present invention to provide a new and improved mitten restraint device that has all of the advantages of the prior art and none of the disadvantages.

It is another object of the present invention to provide a mitten restraint device providing a hand covering in which the user's entire hand can be positioned.

Another object of the present invention is to provide a mitten restraint device having a padded lower portion and a mesh upper portion for allowing the user and healthcare professionals to see the user's hand and fingers while the user is wearing the mitten restraint.

A further object of the present invention is to provide a mitten restraint device having padding for limiting the mobility of a user's hand and fingers.

Yet another object of the present invention is to provide a mitten restraint device having an adjustable strap for securing the mitten restraint on the hand of the user.

Another object of the present invention is to provide a mitten restraint device having a movable flap for removably covering the fingertips of the user.

A further object of the present invention is to provide a mitten restraint device having finger compartments in which the user's fingers can be positioned in order to further restraint the movement of the user's hand, thus preventing rotation of the user's fingers.

Another object of the present invention is to provide a mitten restraint device having a securement strap on the interior thereof that is adapted to be disposed over a portion of the user's hand in order to help restraint the movement of the user's hand.

An additional object of the present invention is to provide a mitten restraint device that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the mitten restraint device.

FIG. 2 shows a side cross sectional view of the mitten restraint device.

FIG. 3 shows a perspective view of the mitten restraint device in use.

### DETAILED DESCRIPTION OF THE INVENTION

Reference is made herein to the attached drawings. Like reference numerals are used throughout the drawings to depict like or similar elements of the mitten restraint device. For the purposes of presenting a brief and clear description of the present invention, the preferred embodiment will be discussed as used for restraining the hand of a user to prevent the user from removing tubes and lines from his or her body, and/or to limit the movement of the user's hand and fingers. The figures are intended for representative purposes only and should not be considered to be limiting in any respect.

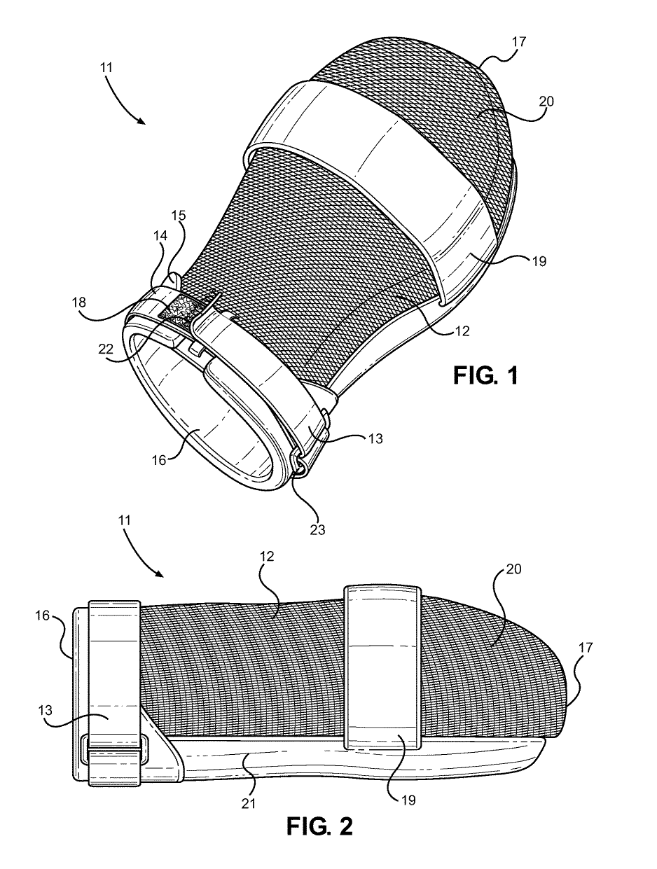

Referring now to FIG. 1, there is shown a perspective view of the mitten restraint device. The mitten restraint device 11 comprises a hand covering for restraining the motion of a user's hand and fingers. The mitten restraint device comprises a hand covering having an opening 16, a lower portion 13, and an upper portion 12. The user can insert his or her hand into the hand covering via the opening 16 and can adjustably secure the hand covering thereon via an adjustable strap. The interior volume of the mitten restraint device 11 comprises a securement strap 19 adapted to extend across the user's hand inserted within the mitten restraint device 11, and further includes a plurality of finger compartments 21 for individually restraining the user's fingers. A movable flap 20 is located on the tip 26 of the mitten restraint device 11 and can be movably positioned so as to expose or cover the user's fingertips.

The mitten restraint device 11 is adapted to receive a user's hand therein, wherein the hand covering comprises an opening 16 thereon for providing access to the interior volume of the hand covering. The interior volume of the hand covering is adapted to receive a user's entire hand therein so that the hand covering substantially covers the user's thumb and fingers. The hand covering comprises a semi-rigid construction so as to limit the user's ability to bend or curl his or her hand or fingers. The hand covering comprises an upper portion 12 that extends over the back of a user's hand and a lower portion 13 on which the palm of the user's hand can rest. The upper portion 12 includes a mesh-like material. The mesh-like material allows the user's hand to be visible while inserted in the hand covering. Further, the mesh-like material allows for air to circulate inside of the hand covering so that the user's hand does not become sweaty or uncomfortable. The lower portion 13 of the hand covering includes a padded material that helps to cushion the user's hand while also restricting the movement of the user's fingers. The padded material is preferably constructed so as to limit the movement of the user's hand while inside of the mitten restraint device 11.

The opening 16 of the hand covering includes padding 15 therearound. The padding 15 helps to provide cushioning to the user's wrist. Further, an adjustable strap having a first section 14 and a second section 17 can be secured by overlapping the first section 14 with the second section 17. The first and second sections 14, 17 include mating fasteners 18, 22 thereon for securing the adjustable strap in an overlapping fashion. Preferably, the fasteners 18, 22 include hook and loop fastening material so that the strap can be easily secured to achieve a desired fit on users of various sizes.

Referring now to FIG. 2, there is shown a side cross sectional view of the mitten restraint device. The interior of the mitten restraint device 11 comprises a securement strap 19 extending from a first side of the mitten restraint device 11 to an opposing side thereof. Preferably, when the user's hand is inserted within the mitten restraint device 11, the securement strap 19 extends across the user's hand. Thus, the securement strap 19 is positioned orthogonally to the longitudinal axis of the user's arm and hand. The securement strap 19 helps to restrain the user's hand within the mitten restraint device 11 so that the user cannot easily move his or her hand.

Further, the interior volume of the mitten restraint device 11 comprises a plurality of finger compartments 21. Preferably, each finger has its own finger compartment 21. The finger compartments 21 are formed by stitching a panel of fabric to the lower portion 13 of the mitten restraint device 11. In this way, tubular finger compartments 21 are formed, wherein the user's finger can extend through the finger compartment 21 and the fingertip extends out of the finger compartment 21.

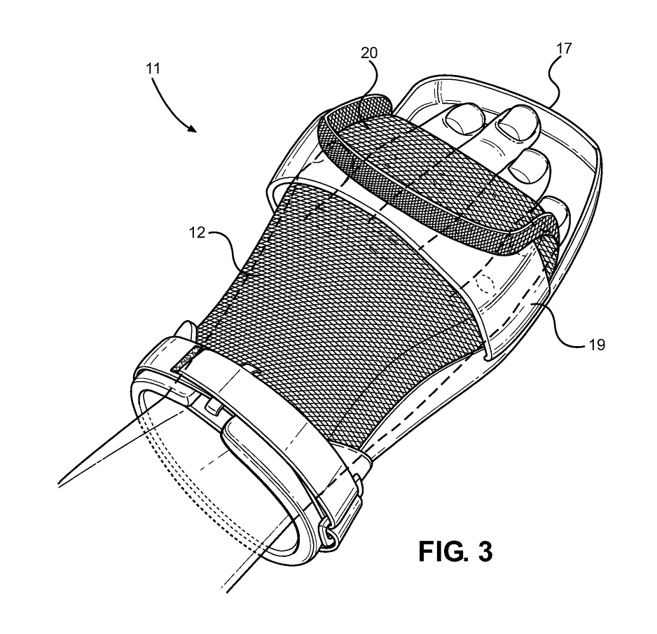

Referring now to FIG. 3, there is shown a perspective view of the mitten restraint in use. The tip 26 of the mitten restraint device 11 comprises a movable flap 20 thereon. The flap 20 comprises a pocket shaped member adapted to cover the upper portion of a user's fingers. The flap 20 is preferably an integral part of the upper portion 12 of the hand covering. An end of the flap 20 comprises a fastener 27 adapted to removably engage with a mating fastener 28 disposed on the underside of the lower portion 13 of the mitten restraint device 11. Preferably, the fasteners 27, 28 comprise hook and loop fastening material, however, alternate types of fasteners such as snaps are used in other embodiments. In this way, the movable flap 20 can be secured in a position wherein the movable flap 20 covers the user's fingertips. Alternatively, the fasteners 27, 28 can be disengaged so that the movable flap 20 can be folded away from the user's fingers and towards the upper portion 12 of the hand covering in order to reveal the user's fingertips. This allows a healthcare professional or the user to have access to the user's fingertips as necessary to perform various tests or to examine the user's fingers.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A restraint mitten, comprising:
a hand covering having an opening, a lower portion, and an upper portion, wherein said hand covering is adapted to receive a user's thumb and fingers therein via said opening;
an adjustable strap disposed around said opening;
said upper portion comprising a mesh material;
said upper portion further comprising a flap adapted to removably cover said fingers of a user.

2) The restraint mitten of claim 1, wherein said lower portion comprises a padded material.

3) The restraint mitten of claim 1, wherein said lower portion is semi-rigid, such that it is adapted to restrain the movement of a user's hand.

4) The restraint mitten of claim 1, wherein said opening includes padding therearound.

5) The restraint mitten of claim 1, further comprising a securement strap that extends across said upper portion in an area corresponding to a location of said user's fingers.

6) The restraint mitten of claim 1, wherein said flap comprises a pocket shaped member adapted to receive said user's fingers therein, and wherein said flap can be arranged so as to cover or reveal said user's fingers.

7) The restraint mitten of claim 6, wherein said flap further comprises a fastener thereon adapted to removably engage with a mating fastener disposed on said lower portion.

8) The restraint mitten of claim 7, wherein said fastener and said mating fastener comprises hook and loop fastening material.

9) The restraint mitten of claim 1, further comprising a plurality of finger compartments within said hand covering.
